(19) United States
(12) Patent Application Publication
(10) Pub. No.: US 2021/0252229 A1
(43) Pub. Date: Aug. 19, 2021

(54) UNIVERSAL SMART CAP FOR PEN INJECTORS

(71) Applicant: Becton, Dickinson and Company, Franklin Lakes, NJ (US)

(72) Inventors: Amit LIMAYE, Wayne, NJ (US); Jeremy GARTNER, Closter, NJ (US); Philip PONCE DE LEON, Franklin Lakes, NJ (US)

(73) Assignee: Becton, Dickinson and Company, Franklin Lakes, NJ (US)

(21) Appl. No.: 17/274,049

(22) PCT Filed: Aug. 29, 2019
(86) PCT No.: PCT/US19/48748

§ 371 Date: Mar. 5, 2021

Related U.S. Application Data
(60) Provisional application No. 62/738,202, filed on Sep. 28, 2018.

(51) Int. Cl. A61M 5/315; A61M 5/32; G16H 20/17; G16H 40/67

(52) U.S. Cl. CPC A61M 5/31568; A61M 5/3202; G16H 20/17; G16H 40/67; A61M 2005/3126; A61M 2205/14; A61M 2205/3584; A61M 2205/3306; A61M 2205/18; A61M 2205/50

(57) ABSTRACT

A two-part cap (100) for an injection pen (106) is provided. The cap includes a static portion (102) that remains on the pen injector (106), and a removable portion (104). The static portion (102) includes a dose sensor (126) for sensing movement of a plunger (118) within a reservoir (114) of the pen injector (106). A delivered dose is calculated based on the detected plunger movement.

### PRIORITY CLAIM

This application claims the benefit under 35 U.S.C. 119 of U.S. Provisional Application Ser. No. 62/738,202, filed Sep. 28, 2019, the entire contents of which is hereby incorporated by reference.

### FIELD OF THE INVENTION

The present invention relates generally to medicine dose measurement devices. More particularly, the present invention relates to a cap for a pen injector that directly detects plunger movement and measures doses during an injection.

### BACKGROUND OF THE INVENTION

Diabetes is a group of diseases marked by high levels of blood glucose resulting from defects in insulin production, insulin action, or both. Diabetes can lead to serious health complications and premature death, but there are well-known products available for people with diabetes to help control the disease and lower the risk of complications.

Treatment options for people with diabetes include specialized diets, oral medications and/or insulin therapy. The primary goal for diabetes treatment is to control the patient's blood glucose (sugar) level in order to increase the chances of a complication-free life. It is not always easy, however, to achieve good diabetes management, while balancing other life demands and circumstances.

Pen injectors have traditionally provided people who require insulin therapy with a convenient, portable means of injecting insulin. However, in order for diabetes management via insulin pen to be successful, it is important that patients administer doses of medication as directed by their medical provider and also give themselves injections properly. The insulin pen should be held in place by the patient for a period of time immediately following injection, so that the insulin is properly absorbed by the body. Health care providers also wish to have an accurate record of injection amounts and times to assist them in evaluating the patient's diabetes management. Presently these functions are performed manually. The patients are instructed to inject the medication and count to a certain number, for example to 10, to ensure that the entire dose of medication has been absorbed into the skin and delivered to the patient. Similarly, following injection, patients are expected to keep a record of the amount of insulin injected and time of injection. Existing pen injectors are plentiful, but do not assist users with proper injection technique, and do not record or communicate dose records to a healthcare provider. Accordingly, there is a need for a device to assist users in properly injecting insulin and for recording successful or unsuccessful doses and communicating that information to a user and their healthcare provider.

### SUMMARY OF THE INVENTION

An aspect of illustrative embodiments of the present invention is to substantially address the above and other concerns, and provide a replacement cap for an injection pen. The replacement cap includes a static part that remains on the injection pen during injections, and a removable cap that covers the distal end of the injection pen when not in use, and is removed for use. The static part of the cap includes electronics and sensors to detect the position and movement of a plunger within a vial of medication, and records doses based on the detected movement.

Additional and/or other aspects and advantages of the present invention will be set forth in the description that follows, or will be apparent from the description, or may be learned by practice of the invention. The present invention may comprise a method or apparatus or system having one or more of the above aspects, and/or one or more of the features and combinations thereof. The present invention may comprise one or more of the features and/or combinations of the above aspects as recited, for example, in the attached claims.

### BRIEF DESCRIPTION OF THE DRAWINGS

The various objects, advantages and novel features of illustrative embodiments of the present invention will be more readily appreciated from the following detailed description when read in conjunction with the appended drawings, in which:

FIGS. 1-3 are side views of an exemplary embodiment of the invention;

FIG. 4 is a block diagram of an exemplary embodiment of the invention;

FIG. 5 is a flow chart of a method according to an exemplary embodiment of the invention;

FIGS. 6-8 are screenshots of a user interface on a mobile device according to an exemplary embodiment of the invention;

FIG. 9 is a system diagram of an exemplary embodiment of the invention;

FIG. 10 is a perspective view of an exemplary embodiment of the invention;

FIG. 11 illustrates front and cross sectional views of a cap according to an exemplary embodiment of the invention; and

FIG. 12 illustrates side and cross sectional views of a cap according to an exemplary embodiment of the invention.

Throughout the drawings like reference numbers will be understood to refer to like features, elements and structures.

### DETAILED DESCRIPTION OF THE ILLUSTRATIVE EMBODIMENTS

As will be appreciated by one skilled in the art, there are numerous ways of carrying out the examples, improvements, and arrangements of a smart cap for a pen injector in accordance with embodiments of the present invention disclosed herein. Although reference will be made to the illustrative embodiments depicted in the drawings and the following descriptions, the embodiments disclosed herein are not meant to be exhaustive of the various alternative designs and embodiments that are encompassed by the disclosed invention, and those skilled in the art will readily appreciate that various modifications may be made, and various combinations can be made, without departing from the invention.

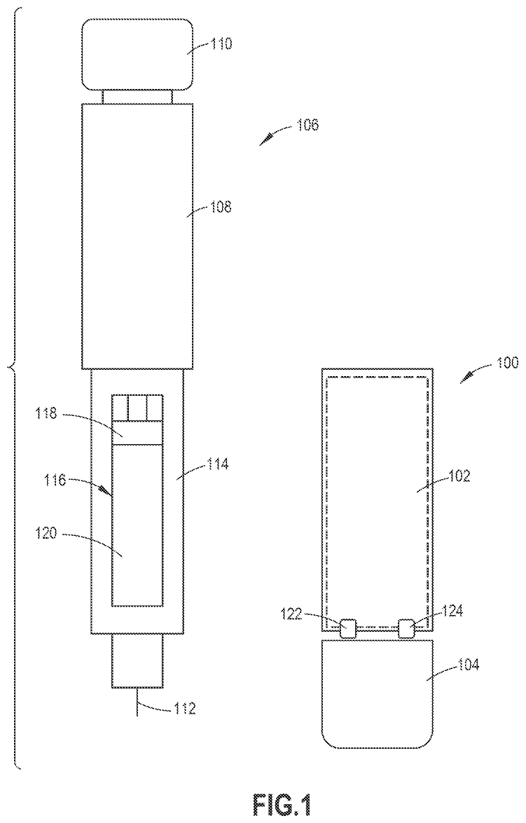

As illustrated in FIG. 1, an exemplary embodiment of the invention comprises a two-part cap 100 for a pen injector. The cap 100 includes a static portion 102 and a removable portion 104. The cap 100 may be fitted onto a pen injector 106. The pen injector components include a pen injector body 108, a thumb button 110, a disposable pen needle 112 and a reservoir compartment 114. Reservoir compartment 114 also includes a window 116 through which a plunger 118 and drug fluid 120 remaining in a reservoir installed in the reservoir compartment 114 may be viewed.

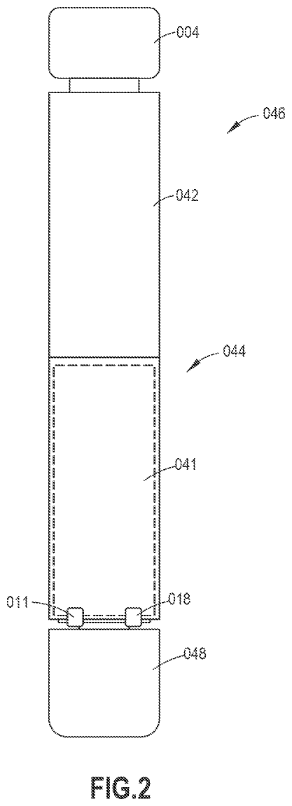

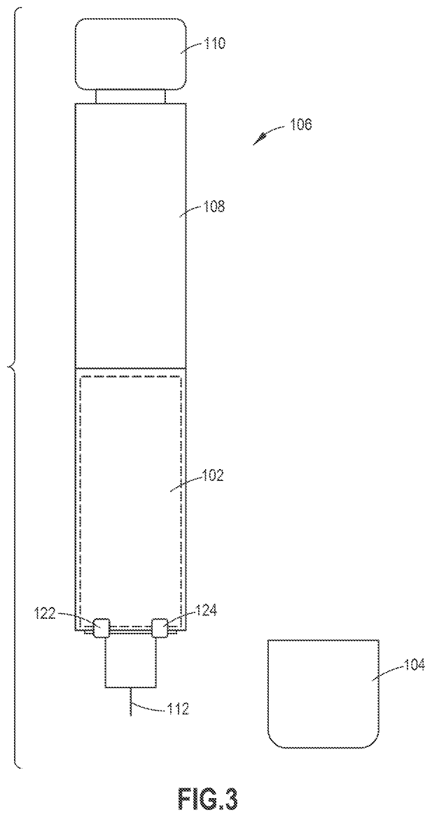

FIG. 2 illustrates the cap 100 installed onto the pen injector 106. FIG. 3 illustrates the removable portion 104 of the cap 100 being removed to expose the pen needle 112 and permit an injection. Removable portion 104 preferably connects and disconnects from the static portion 102 by friction fit, snap fit, threading, or any other suitable mechanism.

In exemplary embodiments of the invention, the static portion 102 of the cap 100 includes three sensors. There is a cap sensor 122 to sense the presence of absence of the removable portion 104 of the cap 100. There is also a proximity sensor 124 which senses whether the pen injector remains pressed against the skin of a user during an injection. The third sensor is a dose sensor 126 that is incorporated into the static portion 102 of the cap 100, and will be described in further detail below. The dose sensor 126 advantageously senses the plunger location and/or remaining fluid in the reservoir, either optically or by any other suitable means. Because the static portion 102 of the cap 100 remains on the pen injector 106 during an injection, the dose sensor 126 can track the movement of the plunger 118 in real time during an injection.

As illustrated in the exemplary embodiment shown in FIG. 3, the removable portion 104 of the cap 100 can be sized to accommodate the pen needle 112. In other embodiments, the removable portion 104 of the cap 100 may be sized not to accommodate a pen needle, so that pen needle is attached and detached from the pen injector 106 after the removable portion 104 is removed and before the removable portion 104 is replaced.

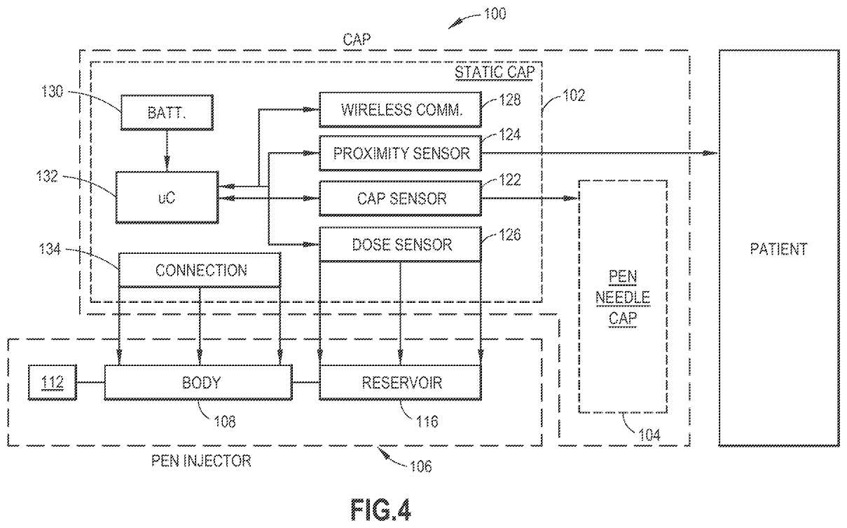

FIG. 4 is a block diagram of the cap 100 and pen injector 106 described above. Cap 100 includes static cap 102 and removable pen needle cap 104. Static cap 102 houses a cap sensor 122, proximity sensor 124 and dose sensor 126, as described above. The static cap 102 also houses a wireless communication unit 128 for communicating with a remote device such as a mobile unit, as will be discussed in further detail below. The static cap houses a power supply battery 130 and a microcontroller 132 for receiving signals from the sensors and controlling communications with remote devices via the wireless controller 128, among other functions. The static cap 102 preferably includes mechanical connection features 134 to connect the static cap to the body 108 of the pen injector 106.

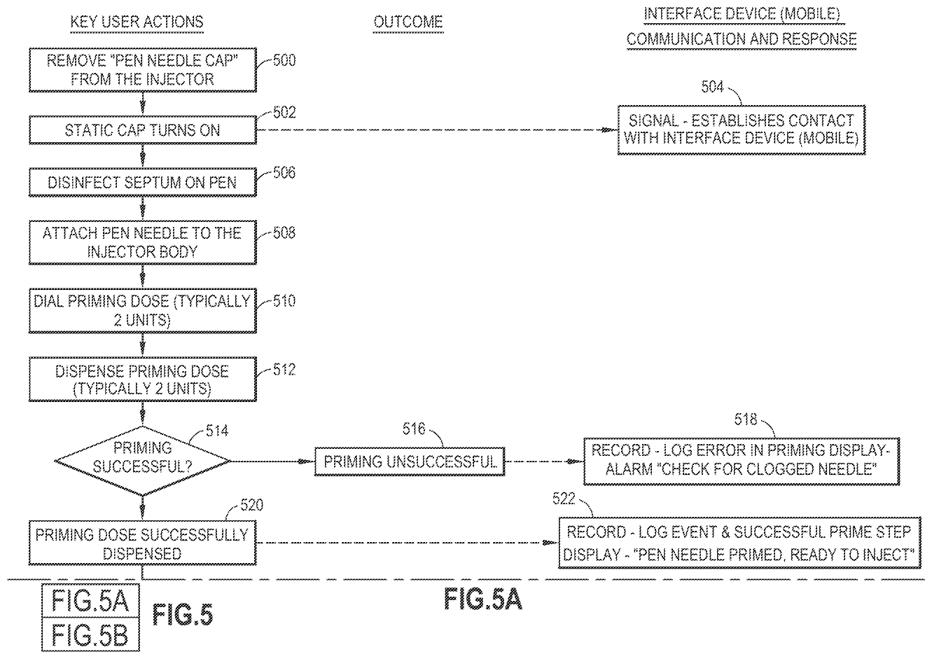

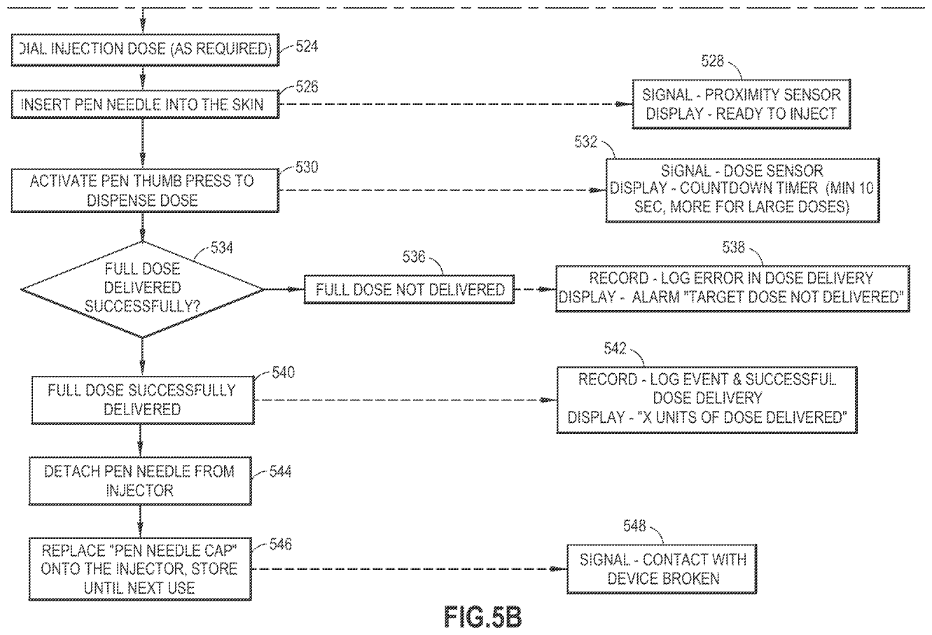

FIG. 5 illustrates an exemplary method of using the cap 100 in connection with the pen injector 106. At step 500, the removable pen needle cap 104 is removed from the injector 106. At step 502, the cap detector 122 detects that the removable cap has been removed, and wakes the static cap 102, the static cap 102 turns on and preferably at step 504, the static cap 102 establishes contact with a remote device such as a mobile phone or the like. At step 506, the user disinfects the septum on the pen. At step 508, the pen needle 112 is attached to the pen injector body 108. At step 510, a user dials a priming dose, which is typically two units. At step 512, the user dispenses the priming dose. At step 514, the user determines if priming was successful. If priming was unsuccessful, the method continues at step 516 and at step 518 an error in priming is logged and an alarm to check for a clogged needle is displayed to the user on the connected mobile device. If priming was successful, then the method continues to step 520 and the device records and logs the successful priming step as an event at step 522 and displays “pen needle primed ready to inject” on the mobile device. Next, the user dials a dose to be injected at step 524. At step 526 the pen needle is inserted into the skin of the patient. The proximity sensor 124 detects that the pen needle has been inserted into the skin and displays “ready to inject” on a display of the connected mobile device at step 528. At step 530, the user activates the pen with a thumb press on the button to dispense the dose and at step 532 the dose sensor 126 senses that the dose has been activated and the display displays a countdown timer preferably for a minimum of 10 seconds or more for larger doses. The dose sensor 126 preferably senses and records movement data corresponding to plunger movement, and records a dose calculated based on the plunger movement. At step 534 device determines if a full dose was successfully delivered. If the full dose was not delivered, the method continues to step 536 and at step 538, an error in dose delivery is recorded in an electronic log and displays “target does not delivered”. If the dose was successfully delivered, then the method continues to step 540 and at step 542 the device records the successful dose delivery and displays “x units of dose delivered” on the display of the mobile device. At step 544, the user detaches the pen needle from the injector. At step 546, the user replaces the pen needle cap 104 onto the pen injector 106 and stores it until the next use. Preferably at this point, at step 548, the electronics of the static cap 102 recognize that contact with the mobile device has been broken and records that event.

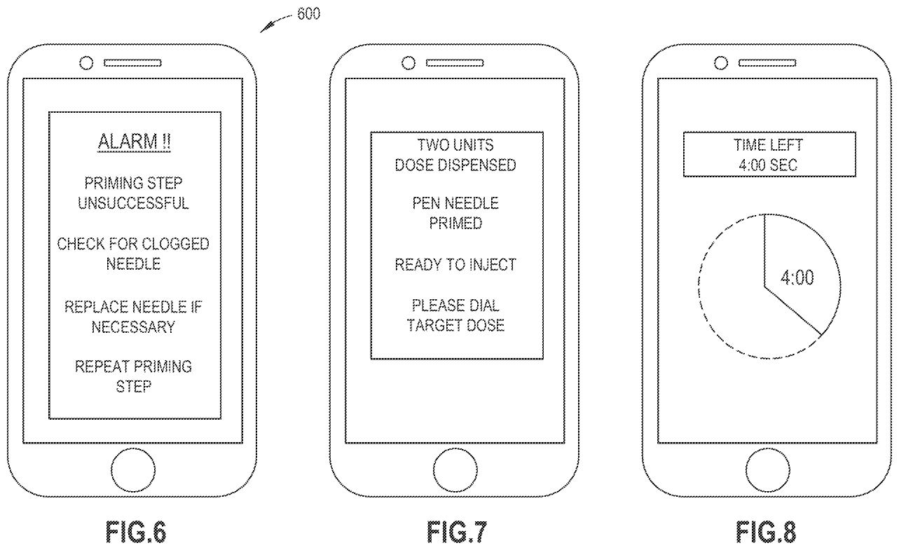

FIGS. 6-8 are exemplary user interface displays on a mobile device 600 according to an exemplary embodiment of the invention. It should be appreciated by those of ordinary skill in the art the that the messages depicted herein are merely exemplary, and that alternate messages or symbols conveying similar meaning could be substituted. FIG. 6 illustrates an exemplary display for use when the priming step is unsuccessful. The display reads “ALARM!! Priming Step Unsuccessful. Check for Clogged Needle. Replace Needle of Necessary. Repeat Priming Step.” FIG. 7 illustrates an exemplary display for use when the priming step is successful. The display reads “Two Units Dose Dispensed. Pen Needle Primed. Ready to Inject. Please Dial Target Dose.” FIG. 8 illustrates an exemplary display for use during the injection. The display shows a countdown timer that preferably counts down from ten second, during which the user should keep the pen injector pressed against the skin. The proximity sensor 124 confirms that the pen injector 106 is not removed prematurely, and causes an error if the pen injector 106 is removed prematurely.

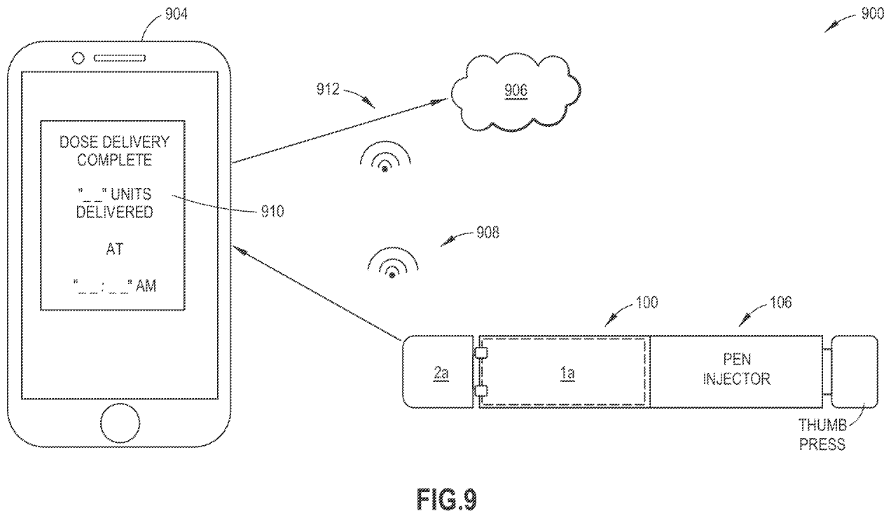

FIG. 9 illustrates a system 900 according to an exemplary embodiment of the invention. The system 900 comprises a pen injector 106 with a cap 100 substantially as described above, a mobile device 904 and a remote server or cloud storage 906. The dose sensor 126 and proximity sensor 124 of the cap 100 preferably transmit signals to the mobile device 904, via wireless communication link 908. In this manner the mobile device can record and analyze events taking place with the add-on device and pen injector 902, and additionally provide feedback to a user on a display 910 of the mobile device 904. The mobile device 904 in turn advantageously has a wireless communication link 912 to a remote server 906, which may be cloud storage, or the like. The mobile device 904 preferably transmits information to the remote server so that the information may be accessed by a healthcare provider or other relevant party. The information transmitted to the remote server 906 may be all of the data received by the mobile device 904 from the pen injector 106 and cap 100, or preferably may be a subset of the information, or results and/or summaries of information received by the mobile device 904 and analyzed according to program instruction installed in the mobile device 904.

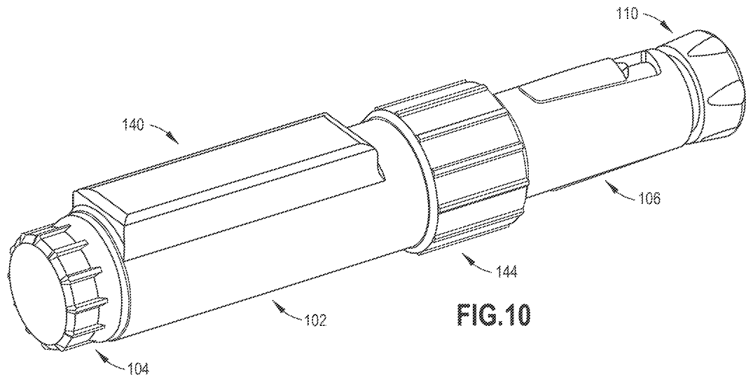

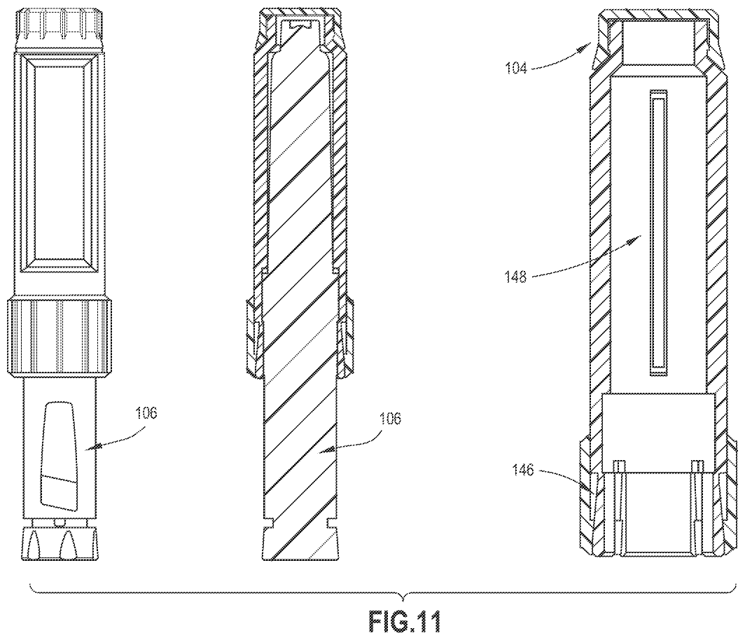

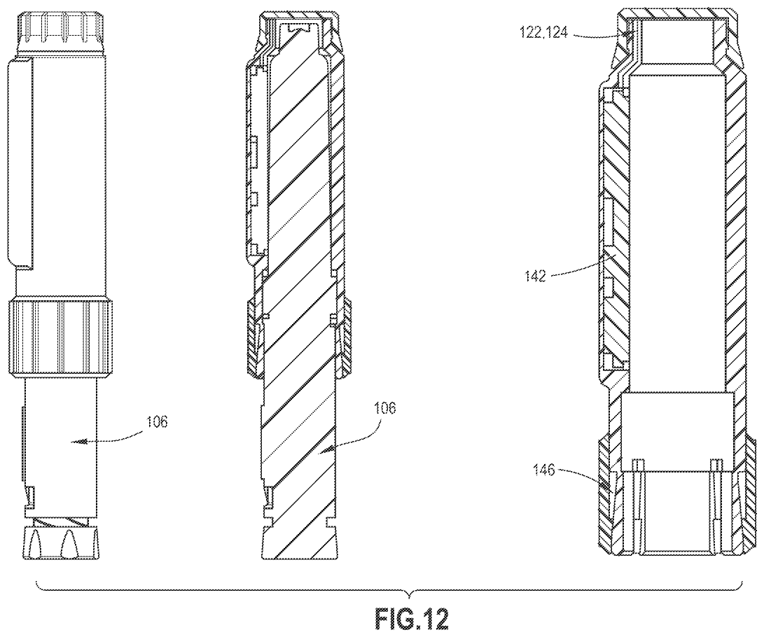

FIG. 10 is a perspective view of an embodiment of the present invention. As illustrated, the cap 100 includes an electronics housing 140. The electronics housing 140 houses an electronics board 142 as illustrated in side view FIG. 12. Cap 100 also includes a locking nut 144 to secure the cap 100 to the pen injector 106. FIG. 11 is a front view of the cap 100, illustrating a collet or chuck clamping mechanism 146 the works with locking nut 144 to secure the cap 100 to pen injector 106. FIG. 11 also illustrates a sensor array 148 on an inside surface of the cap 100 to sense plunger position and movement during an injection. It should be appreciated that the sensor array may be any suitable sensor, including an optical reflective emitter and sensor arranged on the same side of the pen injector 106 and vial, or a transmissive emitter and sensor arranged on an opposite sides of the vial.

FIGS. 10-12 also illustrate and example of a removable portion 104 sized such that the pen needle 112 is not accommodated within the removable portion 104 while the removable portion 104 is attached to the static portion 102.

Although only a few illustrative embodiments of the present invention have been described in detail above, those skilled in the art will readily appreciate that many modifications are possible in the illustrative embodiments, and various combinations of the illustrative embodiments are possible, without materially departing from the novel teachings and advantages of this invention. Accordingly, all such modifications are intended to be included within the scope of this invention.

1. A dose measuring device (100) for use with a pen injector (106), comprising:
a fixed cap part (102) attached to a body of a pen injector, and a removable cap part (104) attached to the fixed cap part (102), and removable from the fixed cap part (102) to expose a pen needle (112) of the pen injector (106);
the fixed cap part (102) comprising a cap sensor (122) for sensing when the removable cap (104) is removed from the fixed cap part (102), a proximity sensor (124) for sensing when the pen injector (106) is adjacent to a user, and a dose sensor (126) for detecting movement of a plunger (118) within a medicament reservoir (114) during an injection.

2. The dose measuring device of claim 1, wherein the fixed cap part further comprises a wireless communication unit that communicates dose data to a remote device.

3. The dose measuring device of claim 2, wherein the remote device is a mobile phone.

4. The dose measuring device of claim 1, further comprising a microcontroller that receives input signals from the proximity sensor, the cap sensor, and the dose sensor.

5. The dose measuring device of claim 4, wherein the microcontroller is further adapted to determine dose information from the received signals, and to transmit the dose information to a remote device.

6. The dose measuring device of claim 1, wherein the dose sensor comprises a light source and at least one photodetector that receives light transmitted from the light source and through the reservoir.

7. The dose measuring device of claim 1, wherein the dose sensor comprises a light source and at least one photodetector that receives light transmitted from the light source and reflected from the plunger.

8. A method of measuring a dose comprising the steps of:
attaching a cap to a pen injector, the cap comprising a static cap part (102) and a removable cap part (104) removably attached to the static cap part (102), wherein the static cap part (102) comprises a dose sensor (126), a removable cap sensor (122) and a proximity sensor (124),
detecting removal of the removable cap part (104) with the removable cap sensor (122);
waking the static cap part (102) when removal of the removable cap part (104) is detected;
sensing movement of a plunger (118) within the pen injector (106) during an injection;
calculating a dose based on detected movement of the plunger (118); and
transmitting dose information to a remote device.

9. The method of claim 8, further comprising determining, based on sensed movement of the plunger, that a pen needle attached to the pen injector is clogged.

10. The method of claim 9, further comprising communicating an alert to the remote device if a clog is detected.

11. The method of claim 8, further comprising providing a count-down display on the remote device during an injection, and determining if the proximity sensor detects that the pen injector contacts skin of a user throughout an injection.

12. The method of claim 8, further comprising transmitting dose information from the remote device to a cloud storage device.
